(19) United States
(12) Patent Application Publication
(10) Pub. No.: US 2003/0083734 A1
(43) Pub. Date: May 1, 2003

(54) Stent

(75) Inventors: Holger Friedrich, Dresden (DE); Detlef Schneider, Hagen (DE)

Correspondence Address: COLLARD & ROE, P.C., 1077 Northern Boulevard, Roslyn, NY, 11576, US

(73) Assignee: CURATIVE AG

(21) Appl. No.: 10/279,943

(22) Filed: Oct. 24, 2002

(30) Foreign Application Priority Data
Oct. 25, 2001 (DE) 101 52 066.2
Sep. 7, 2002 (EP) 02020101.8

(51) Int. Cl. A61F 2/06

(52) U.S. Cl. 623/1.15

(57) ABSTRACT

A stent for treating vascular stenoses. The stent is insertable into regions of arterial vascular systems close to the ostium. The stent comprises a tubular expandable element made of a mesh fabric which is insertable into the inlet or an outlet of a vessel. The proximal end of the stent is expandable to a trumpet-shaped structure. Using the inventive stent, it is possible in a simple, rapid and safe manner to treat stenoses close to the ostiums of arterial vascular systems so that the entire stenosed region can be covered with a stent.

### BACKGROUND

The invention relates to a stent for the treatment of arterial vascular stenoses in the ostium of the vessel. This stent is especially for use in a branch of the renal artery from the abdominal aorta or in the main trunk of the coronary vessel system.

The development of atheromatosis of the arterial vascular system is marked by deposition and subsequent stenosis of atheromatous substance commonly referred to as plaque in the vessel walls. These deposits lead to progressive stenosis of the vessels and cause a corresponding loss of elasticity accompanied by a narrowing of the lumen. Coronary vessels and usually also the renal arteries are among the organs affected by this form of vascular disease. In the region of the renal arteries, the stenosis can lead to reduced blood supply to the kidneys and also to the development of systemic high blood pressure. Thus, there should be aggressive treatment of renal artery stenosis due to plaque deposits.

The renal arteries extend approximately at right angles on both sides from the main artery, otherwise known as the abdominal aorta. The stenoses typically affect the ostium of the vessel. Thus, the stenoses are found directly at the point where the renal arteries branch off from the main artery. Until now, the invasive treatment comprised expansion by a balloon catheter in a first step wherein this expansion is maintained by introducing stents (vascular supports) in a second step.

Stents usually have the form of tubular basket structures made of wire. They are pressed against the wall of the vessel and remain there. Thus, these stents are intended to perform a splinting function and act as bracing lining for the reopened vessel. They are available as balloon-expandable or self-expandable models, depending on the choice of material.

For example, German Utility Model 9117152 U1, describes a device such as a percutaneous stent arrangement which is self-expanding for holding vessels open and preventing residual stenoses. This device comprises a plurality of bracing wire rings disposed in a zig-zag form on the inner circumference of a flexible sheath and held spaced apart via an axially disposed wire. This wire is welded on to join the bracing wire rings. Because of the aforesaid anatomical nature of stenosis of the renal arteries, these stents must always overlap the ostium of the vessel, which is the entrance to the vessel as viewed from the abdominal aorta. With the renal artery, these stents are therefore placed so that the end of the stent adjacent to the abdominal aorta protrudes by about one centimeter into the abdominal aorta. There, the stents form a crown-shaped projection, which makes it practically impossible to work with a catheter in subsequent procedures in which the catheter extends through these stents. Furthermore, parts of the stents that do not bear snugly against the vascular wall cause swirling in the blood, with the potential for leading to thrombi. In any case, a stenosis in the ostium region cannot be completely eliminated with such a stent arrangement, since the region of the ostium proximal to the abdominal aorta remains uncovered.

International Patent WO 01/21095 teaches a stent for insertion into a bifurcated body lumen. The stent comprises substantially two tubular members of metal mesh fabric, the first having an aperture into which a second stent can be inserted. The first stent is inserted in the bifurcated lumen so that its aperture is positioned at the branch. The second stent is inserted into the branching lumen so that the starting parts of the stents overlap in the bifurcated body lumen.

An unfavorable feature of this stent arrangement is that it causes high flow resistance. Moreover, the introduction of such a multi-part stent arrangement is highly complicated and thus time-consuming. Lengthy surgical times and increased surgical risk are the undesired consequences.

### SUMMARY

Therefore, one object of the invention is to provide a stent for the treatment of stenoses in a vascular branch. This treatment results in the covering of the stenosed vascular segment in the ostium of the vessel. The stent bears snugly against the vascular walls to avoid high flow resistances and swirling of the blood and also to allow easy introduction of the stent in the vascular branch. Furthermore, the design of the stent is also intended to permit subsequent procedures, wherein it is necessary to work through this stent with a catheter.

This stent is especially for use in stenoses of the renal arteries at the ostium of the vessel. It should be possible to adapt the stent to the anatomical conditions and at the same time to reduce the load on the tissue to a minimum.

This object is achieved according to the invention by using a stent comprising a tubular expandable member having a proximal end and a distal end wherein the proximal end flares out in a form of a trumpet shape.

For optimal coverage of the stenosed tissue, the trumpet-shaped flared structure of the expandable member should be formed at an angle a of 45 to 60° relative to the longitudinal axis of the expandable member.

Furthermore, when the expandable member is in its expanded condition, the maximum diameter of the trumpet-shaped flared structure at its proximal end should be at least 20% larger than the diameter of the tubular part of the expandable member. In addition, the expandable member should be constructed from mesh fabric with helically running strands that cross over one another.

In a favorable embodiment, the tissue compatibility is improved by providing a mesh fabric that forms a ring at its proximal end and/or at its distal end when it is in its expanded condition.

In an advantageous self-expanding version, the mesh fabric of the expandable member is made of elastic material or of material with elastic memory properties. For example, the best results have been achieved with mesh fabrics made of Nitinol. However, it is also possible, to make the expandable member from plastically deformable material, which can be expanded via a balloon to be introduced into the expandable member.

When the stent is used in the region of the renal artery it is necessary to ensure that the distal part, which is the part farthest removed from the vessel entrance, is flexible, so that it can conform better to the physiologically induced positional variations of the vascular system in this area. This flexibility is advantageously achieved by providing a mesh fabric having alternating flexible and rigid regions in an axial direction, beginning with a rigid part at the proximal end.

Parts of the mesh fabric are made more flexible by joining the rigid regions of the mesh fabric by between three and five strands.

The exact spatial distribution between rigid and flexible regions can be freely selected to suit the desired application.

When the inventive stent is used for treatment of stenoses in the ostium of a vessel, especially for expanding and bracing a narrowed renal artery, there is complete coverage of the stenosed vascular segment. Because of the inventive construction, the stent bears snugly against the vascular wall, even in the region of the ostium of the vessel. Thus, high flow resistances and swirling in the blood is avoided due to parts of the stent protruding from the vascular wall. Furthermore, the use of the inventive stent also adds the great advantage so that subsequent procedures are possible on the distal side of that particular ostium.

### BRIEF DESCRIPTION OF THE DRAWINGS

Other objects and features of the present invention will become apparent from the following detailed description considered in connection with the accompanying drawings which disclose at least one embodiment of the present invention. It should be understood, however, that the drawings are designed for the purpose of illustration only and not as a definition of the limits of the invention.

In the drawings, wherein similar reference characters denote similar elements throughout the several views:

FIG. 1 shows a schematic diagram of the stent in its expanded condition;

FIG. 2 shows a schematic diagram of the stent in its collapsed condition;

FIG. 3 shows a schematic diagram of the stent inserted into the ostium of a vessel;

FIG. 4 shows a schematic diagram of the insertion instrument;

FIG. 5 shows a schematic sectional diagram of the distal part of the insertion instrument together with a stent whose proximal end is expanded; and

FIG. 6 shows a schematic sectional diagram of the distal part of the insertion instrument together with a stent which is expanded over its entire length.

### DETAILED DESCRIPTION

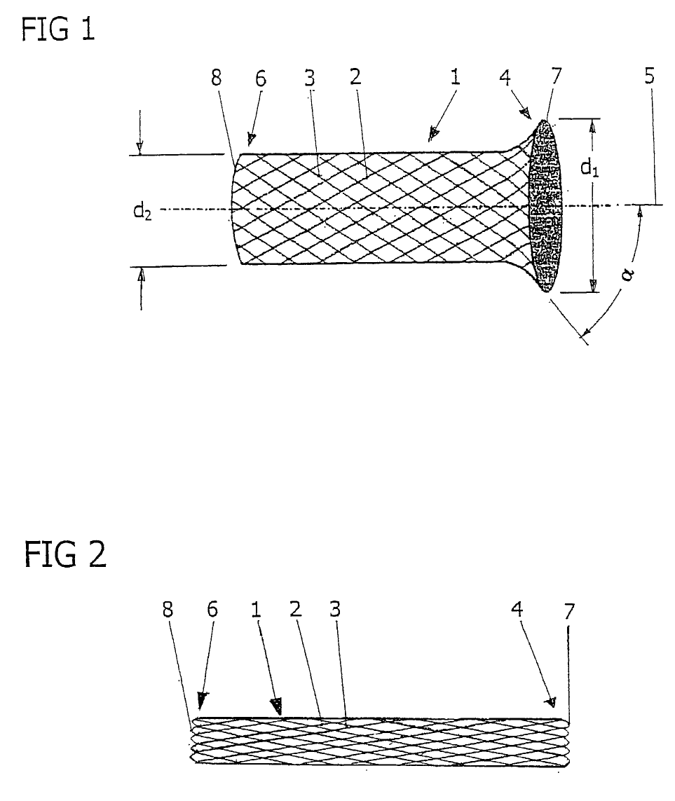

Referring to the drawings, FIGS. 1 and 2 illustrate an inventive stent in the expanded and collapsed condition respectively. The stent comprises a mesh fabric 1 of Nitinol, whose strands 2 and 3 run helically in opposite directions and thus give it a tubular geometry. At its proximal end 4, mesh fabric 1 is flared in the form of a trumpet. The angle a between the longitudinal axis 5 and the trumpet-shaped flared structure is 50°. The largest diameter d₁ of the trumpet-shaped flared structure is 20% larger than the diameter d₂ of the rest of mesh fabric 1.

At its proximal end 4 and distal end 6, mesh fabric 1 is terminated with rings 7, 8 respectively. This means that the ends of strands 2 and 3 are joined to one another so that rings 7 and 8 can be expanded. As illustrated in FIG. 2, mesh fabric 1 of Nitinol can be collapsed to a very small diameter when cold. Upon being heated to body temperature, mesh fabric 1 spreads out by self expansion to its original shape illustrated in FIG. 1, by virtue of the memory capability of its material. The forces developed in the process widen the stenosed vessel and hold it open in a reliable manner.

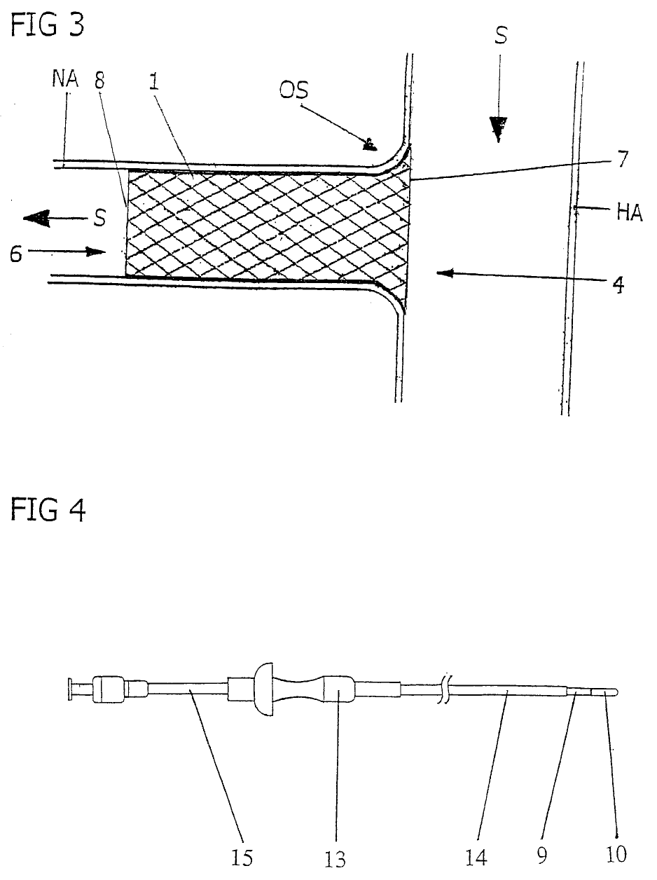

FIG. 3 illustrates the stent inserted in a branch of the renal artery NA extending from the abdominal aorta HA. The trumpet-shaped part of mesh fabric 1, terminated by ring 7, safely surrounds the region of the ostium OS of the renal artery NA without allowing parts of mesh fabric 1 to protrude into abdominal artery HA. In this diagram S denotes the direction of blood flow.

Thus, the stent can be introduced into the vascular segment to be treated in the following manner:

The collapsed mesh fabric 1 illustrated in FIG. 2 is introduced into two sheaths disposed in series, namely a proximal sheath 9 and a distal sheath 10. Thus, proximal end 4 of mesh fabric 1, which has the trumpet-shaped flared structure, becomes lodged in proximal sheath 9, while the protruding remainder becomes lodged in distal sheath 10. Sheaths 9 and 10 are introduced into the vessel to be treated. These sheaths can be introduced via the abdominal aorta HA into the branch of the renal artery NA by means of an insertion instrument illustrated in FIG. 4. The insertion instrument has a manipulating handle 13, to which lining 14 is attached.

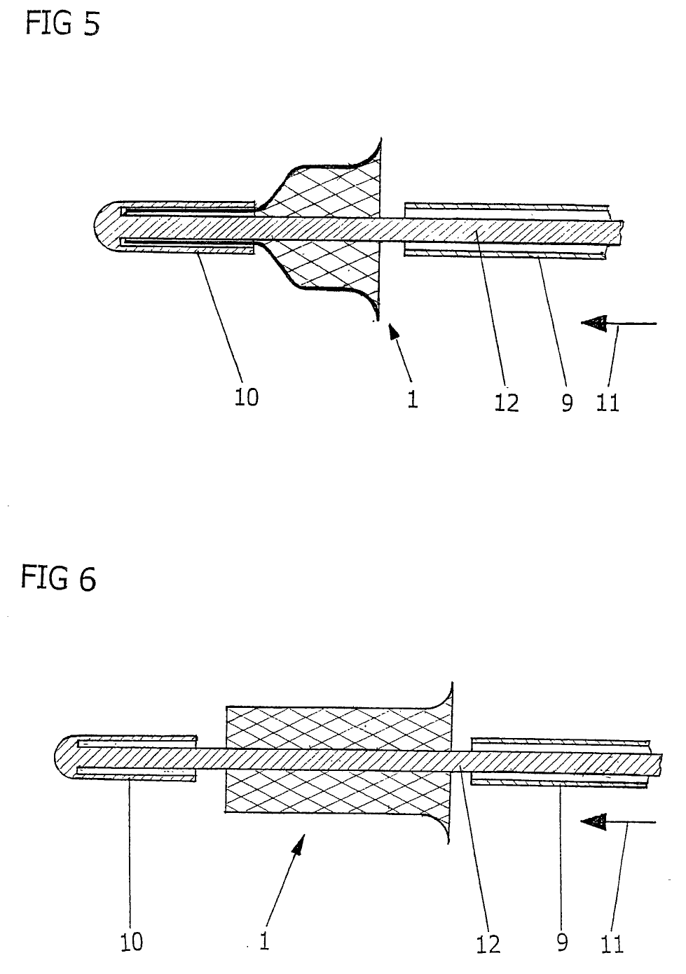

Manipulating handle 13 allows a pusher 15 to pass there through to allow sheath 9 to be displaced. Pusher 15 has a core 12 disposed inside, wherein the core is guided therein and which is joined to distal sheath 10. Once the stent comprising mesh fabric 1 has been correctly positioned at the stenosed segment of the aorta, proximal sheath 9 is pulled off from mesh fabric 1 in the direction opposite to arrow 11, as illustrated in FIG. 5. As a result, the proximal part of mesh fabric 1, containing the trumpet-shaped flared structure, becomes free and thus able to expand. At this time, the distal part of mesh fabric 1 remains in distal sheath 10. Pusher 13, acting via sheath 9 allows mesh fabric 1 to be in a partly expanded form and to be pressed in the direction of arrow 11 firmly into the vascular branch. Thus, the trumpet-shaped flared structure of mesh fabric 1 bears snugly against the ostium of the vessel. Thereafter, distal sheath 10 can be drawn off from mesh fabric 1 in the direction of arrow 11 using core 12 of the insertion instrument, so that the distal part of mesh fabric 1 can now also expand. As a result, the stent assumes the position illustrated in FlG. 3. As illustrated in FIG. 6, distal sheath 10 and proximal sheath 9 together with the entire insertion instrument can now be removed from the vascular system in the direction of arrow 11, through expanded mesh fabric 1.

Accordingly, while at least one embodiment of the present invention has been shown and described, it is to be understood that many changes and modifications may be made thereunto without departing from the spirit and scope of the invention as defined in the appended claims.

What is claimed is:

1. A stent for insertion into a branch of a vessel such as a renal artery from an abdominal aorta comprising:
a tubular expandable member having a proximal end and a distal end wherein said proximal end flares out in a form of a trumpet shape when said tubular expandable member is in an expanded state.

2. The stent as in claim 1, wherein said trumpet shaped flared structure flares out at an angle between 45 and 60 degrees relative to a longitudinal axis of said stent and is at least 20% larger than a diameter of a tubular portion of said tubular expandable member.

3. The stent as in claim 1, wherein said tubular expandable member comprises a mesh fabric having helically running strands that cross over one another.

4. The stent as in claim 3, wherein said proximal end and said distal end are in the form of expanded rings.

5. A stent as in claim 3, wherein said mesh fabric is made from self expanding material having memory properties.

6. The stent as in claim 5, wherein said mesh fabric is made from Nitinol.

7. The stent according to claim 3, wherein said mesh fabric is made to be self expanding from elastic material.

8. The stent as in claim 3, wherein said mesh fabric is made from plastically deformable material.

9. The stent as in claim 3, wherein said mesh fabric has alternating rigid and flexible regions extending along a direction of the longitudinal axis, wherein said proximal end is constructed as a rigid region.

10. The stent as in claim 9, wherein said flexible regions of said mesh fabric are formed using between three and five strands wherein said strands join said rigid regions of said mesh fabric.
